(19) United States
(12) Patent Application Publication
(10) Pub. No.: US 2018/0360365 A1
(43) Pub. Date: Dec. 20, 2018

(54) AUTONOMOUS CONTINUOUS BLADDER IRRIGATION DEVICES, COLORIMETERS, AND METHODS OF MONITORING BLADDER PRESSURE

(71) Applicants: Arun RAI, Houston, TX (US); Baylor College of Medicine, Houston, TX (US)

(72) Inventors: Naveen Yadav, Houston, TX (US); Arun Rai, Houston, TX (US)

(73) Assignee: Baylor College of Medicine, Houston, TX (US)

(21) Appl. No.: 15/780,389

(22) PCT Filed: Dec. 21, 2016
(86) PCT No.: PCT/US16/67918

§ 371 Date: May 31, 2018

Related U.S. Application Data
(60) Provisional application No. 62/387,115, filed on Dec. 23, 2015.

(51) Int. Cl. A61B 5/20; A61B 5/103; A61B 5/00

(52) U.S. Cl. CPC A61B 5/205; A61B 5/1032; A61B 5/6853; A61M 3/0216; A61B 5/0084; A61B 5/6874; A61M 2210/1085

(57) ABSTRACT

One aspect of the invention provides an autonomous continuous bladder irrigation device including: a color sensor programmed to detect a color of outflow from a catheter; an occlusion unit programmed to adjustably occlude a lumen providing irrigation to the catheter; and a controller in communication with the color sensor and the occlusion unit and programmed to control the occlusion unit to titrate irrigation flow to the catheter based on the color detected by the color sensor in order achieve a specified color of outflow from the catheter.

### CROSS-REFERENCE TO RELATED APPLICATION

This application claims priority to U.S. Provisional Patent Application Ser. No. 62/387,115, filed Dec. 23, 2015. The entire content of this application is hereby incorporated by reference herein.

### BACKGROUND OF THE INVENTION

Hematuria is a significant medical problem frequently seen in the setting of trauma, bladder/prostate surgery, and radiation cystitis. According to the Nationwide Emergency Department Sample (component of the Healthcare Cost and Utilization Project), there were approximately 718,913 visits to the ER between 2006 and 2009 with a primary complaint of hematuria; ultimately costing (in the ED only) over $237 million.

### SUMMARY OF THE INVENTION

One aspect of the invention provides an autonomous continuous bladder irrigation device including: a color sensor programmed to detect a color of outflow from a catheter; an occlusion unit programmed to adjustably occlude a lumen providing irrigation to the catheter; and a controller in communication with the color sensor and the occlusion unit and programmed to control the occlusion unit to titrate irrigation flow to the catheter based on the color detected by the color sensor in order achieve a specified color of outflow from the catheter.

This aspect of the invention can have a variety of embodiments. The device can further include a pressure sensor adapted and configured to couple with a balloon port of the catheter. The controller can be in communication with the pressure sensor and further programmed to control the occlusion unit to cease irrigation flow in response to a pressure measurement.

The occlusion unit can include a movable impingement member adapted and configured to impinge flow through the lumen. The impingement member can be a piston. The impingement member can be a cam. The occlusion unit can include a servo motor or a stepper motor adapted and configured to move the impingement member.

The device can further include a flow rate sensor in communication with the controller. The flow sensor can be adapted and configured to measure a flow rate within the lumen below the occlusion unit.

The color sensor can further include: a housing adapted and configured to surround a portion of a lumen; a light source positioned within a first side of the housing and adapted and configured to apply light to the portion of the lumen; and a photodiode sensor positioned on a second, opposite side of the housing and adapted and configured to detect wavelengths of the light that is not absorbed by a sample within the portion of the lumen.

The housing can further include a first half and a second half rotatably coupled by a hinge.

Another aspect of the invention provides a colorimeter including: a housing adapted and configured to surround a portion of a lumen; a light source positioned within a first side of the housing and adapted and configured to apply light to the portion of the lumen; and a photodiode sensor positioned on a second, opposite side of the housing and adapted and configured to detect wavelengths of the light that is not absorbed by a sample within the portion of the lumen.

This aspect of the invention can have a variety of embodiments. The light source can emit a full spectrum of visible light. The housing can include a first half and a second half rotatably coupled by a hinge. The hinge can be a living hinge.

Another aspect of the invention provides a method of monitoring bladder pressure. The method includes: introducing a fluid into a balloon port of a Foley catheter to inflate a balloon of the Foley catheter while the balloon is naturally resting within the bladder; coupling a pressure sensor to the balloon port; and detecting the pressure exerted on the Foley balloon through a column of fluid in contact with the pressure sensor.

This aspect of the invention can have a variety of embodiments. The fluid can be a substantially incompressible fluid. The fluid can be water.

The method can further include calculating a rate of pressure change.

The method can further include controlling an irrigant inflow rate. The irrigant inflow rate can be controlled by actuation of a pump mechanism. The method can further include applying suction to outflow of the catheter. The suction applied to the outflow of the catheter can evacuate obstructing matter within the catheter or bladder.

The method can further include titrating outflow from and inflow to the catheter. The outflow and inflow can be titrated through a controller to generate a specified pressure using the detected pressure as feedback.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIGS. 1A-1B depict a Foley catheter. FIG. 1A shows a top view of a 3-way Foley catheter (top) and a cross-sectional view of the catheter (bottom) taken along the A-A line.

FIGS. 2A-2D depict a color sensing unit according to embodiments of the invention.

FIG. 3 depicts a pressure sensing unit according to embodiments of the invention.

FIGS. 4A-4D depict an occlusion unit according to embodiments of the invention.

FIG. 5 depicts an autonomous continuous bladder irrigation device according to embodiments of the invention.

FIG. 6 depicts a method of monitoring bladder pressure according to embodiments of the invention.

### DEFINITIONS

The instant invention is most clearly understood with reference to the following definitions:

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

### Continuous Bladder Irrigation

Patients with significant hematuria are often placed on continuous bladder irrigation (CBI) to manage and treat the hematuria. The principle of CBI is that continuous irrigation prevents clot formation, but is not so aggressive as to overdistend the bladder and prevent tissue healing. However, current CBI therapy has a high nursing burden and is very sensitive to inappropriate titration and management.

Given the variety of demands on nursing staff in the current hospital environment, CBI is currently not performed efficiently. Specifically, titration is sometimes not performed in a timely manner, as it requires manual flow rate changes on a minute-to-minute basis for some patients. Additionally, irrigation bags frequently are depleted, which can negatively impact therapy and cause significant pain and damage to a patient's bladder if the outlet tubing subsequently becomes obstructed due to clot formation. This inappropriate management of CBI can lead to significant patient discomfort, morbidity, mortality and hospital accrued costs.

### Use of Foley Cather for Continuous Bladder Irrigation

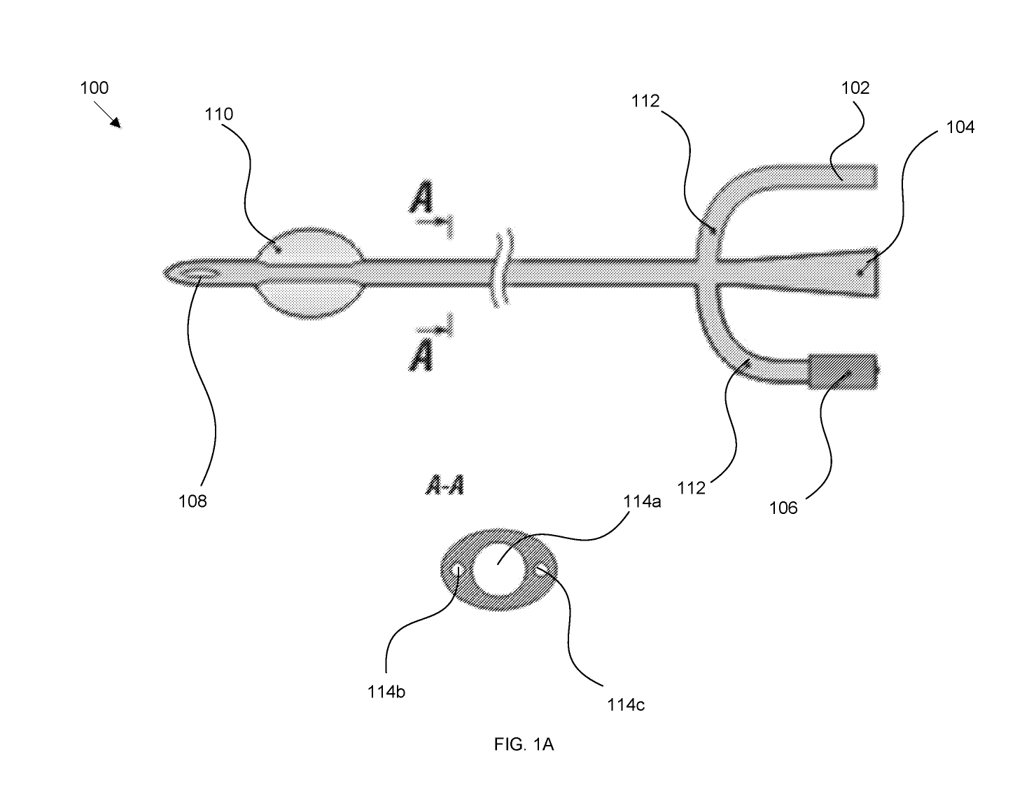

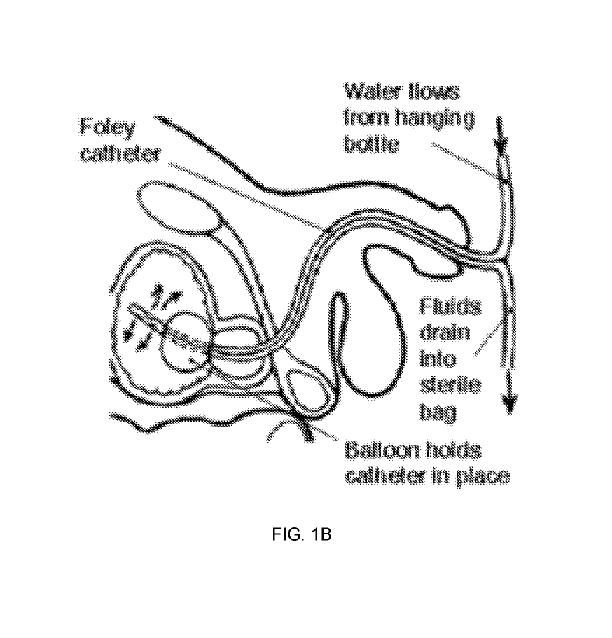

CBI is often performed using a Foley catheter 100 as depicted in FIGS. 1A and 1B. A catheter is a lumen or set of lumens 114a, 114b, 114c that is passed through a patient's urethra to drain urine from the patient's bladder.

A Foley catheter is an indwelling catheter that uses an inflated balloon to keep the catheter in place within the patient. FIG. 1A depicts a 3-way Foley catheter 100. The Foley catheter 100 includes an inlet port 102, an outlet port 104, and a balloon port 106. Lumens 114a, 114b extend from the inlet port 102 and the outlet port 104 through the catheter 100 to one or more openings 108 at a distal end of the catheter 100. A further lumen 114c extends from balloon port 106 to a balloon 110 located at the distal end of the catheter 100. The balloon 110 can be inflated by applying fluid pressure through the balloon port 106 after the distal end of the catheter 100 is inserted into a patient's bladder to retain the distal end of the catheter 100 as depicted in FIG. 1B. For example, sterile water can be introduced to inflate the balloon via a syringe connected to the balloon port 106.

### Autonomous Continuous Bladder Irrigation Device

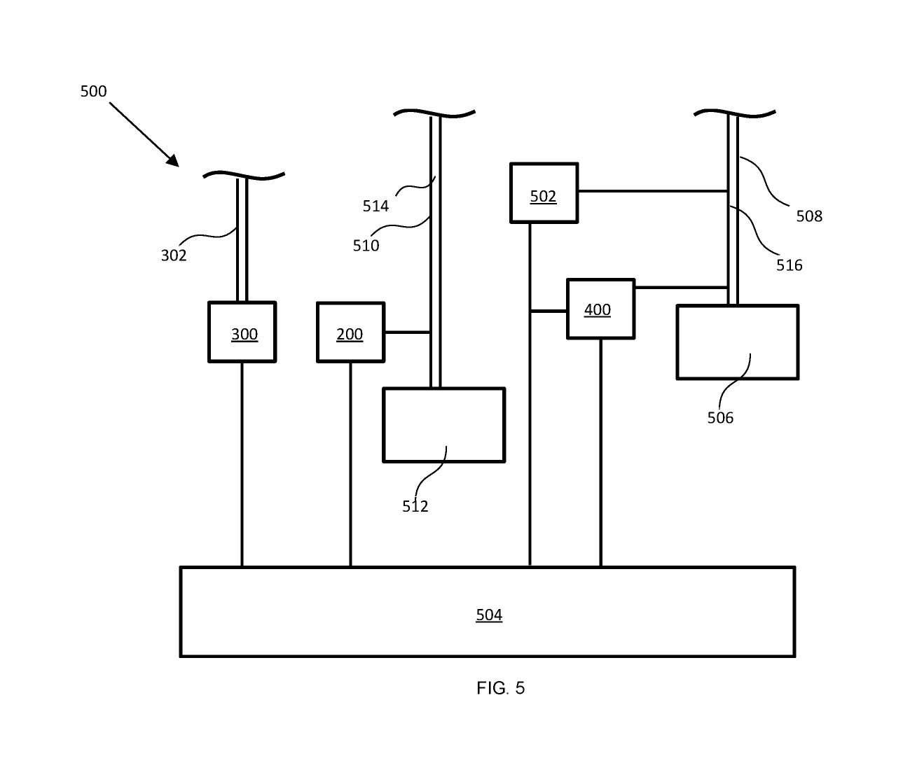

Aspects of the invention provide systems, devices, and methods for continuous bladder irrigation and monitoring bladder pressure. In one aspect, the invention provides an autonomous continuous bladder irrigation device (“ACBID”) 500 as depicted in FIG. 5. The ACBID is a medical device designed to automate all aspects of continuous bladder irrigation (CBI) in a hospital setting. The device 500 is designed to be completely compatible with all existing irrigation tubing, Foley catheters 100, and Foley tubing 112.

In some embodiments, the device includes a color sensing unit 200, an occlusion unit 400, and a controller 504. In some other embodiments, the device 500 further includes a pressure sensing unit 300. In still other embodiments, the device further includes a flow rate sensor 502.

The controller 504 serves as the “brain” of the device 500. The controller 504 can be adapted, configured, and/or programmed to actively receive information from the other portions of the device (e.g., color sensing unit 200, occlusion unit 300) and increase, decrease, or terminate flow to the patient's bladder based on the received information in order to achieve specified (e.g., through programming or user configuration) conditions.

### Color Sensing Unit (“CSU”)

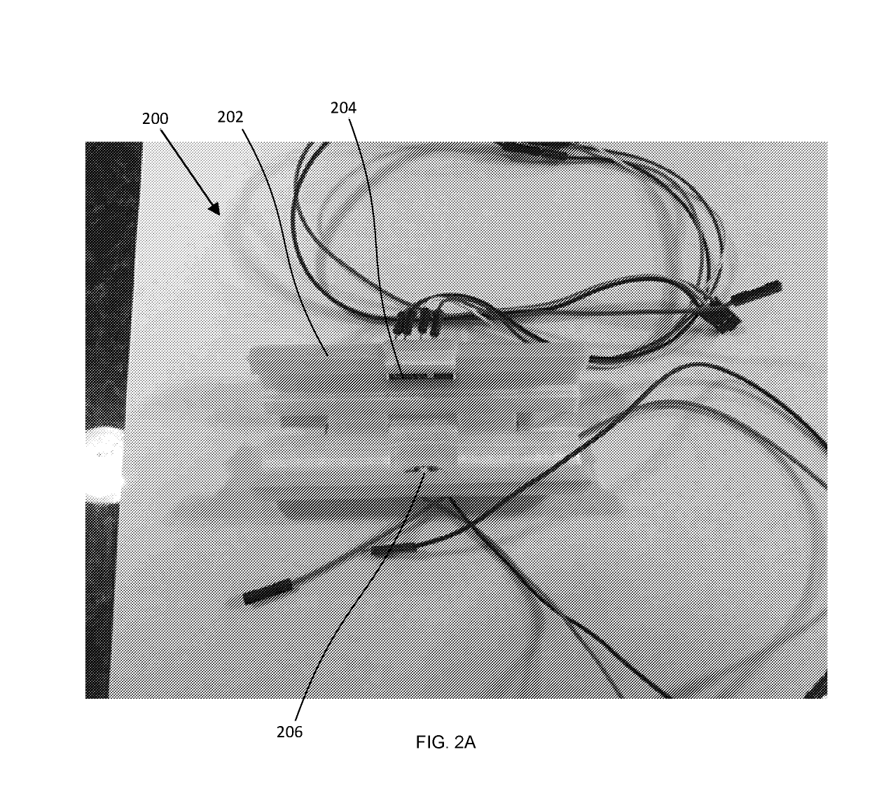

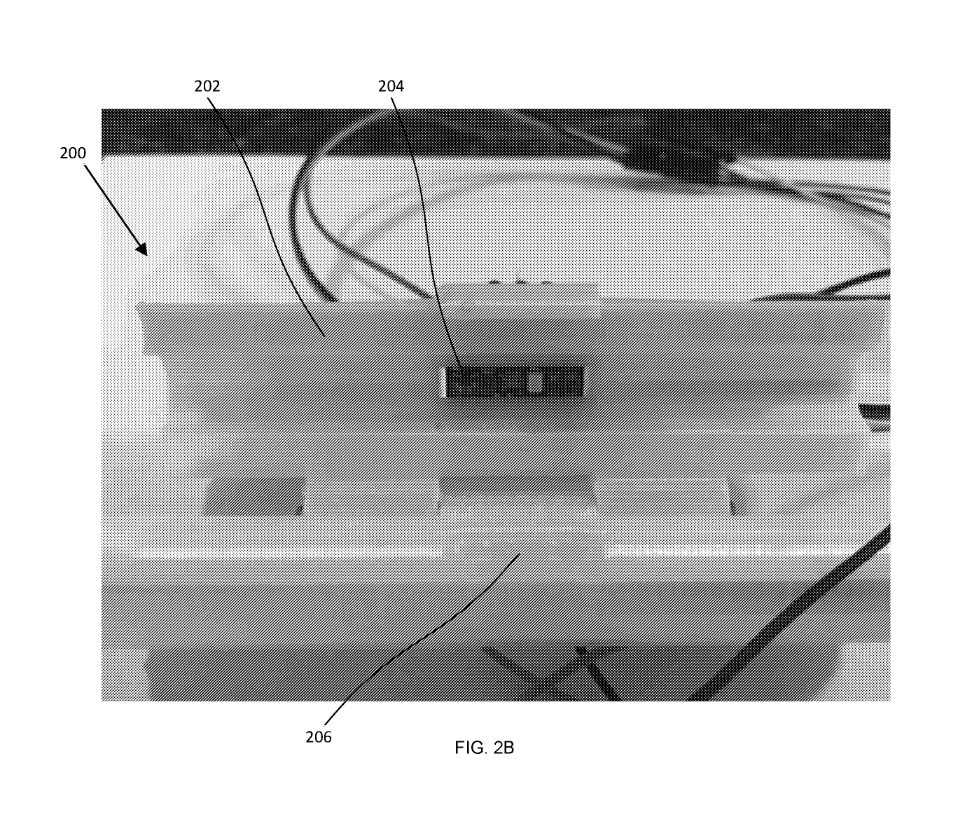

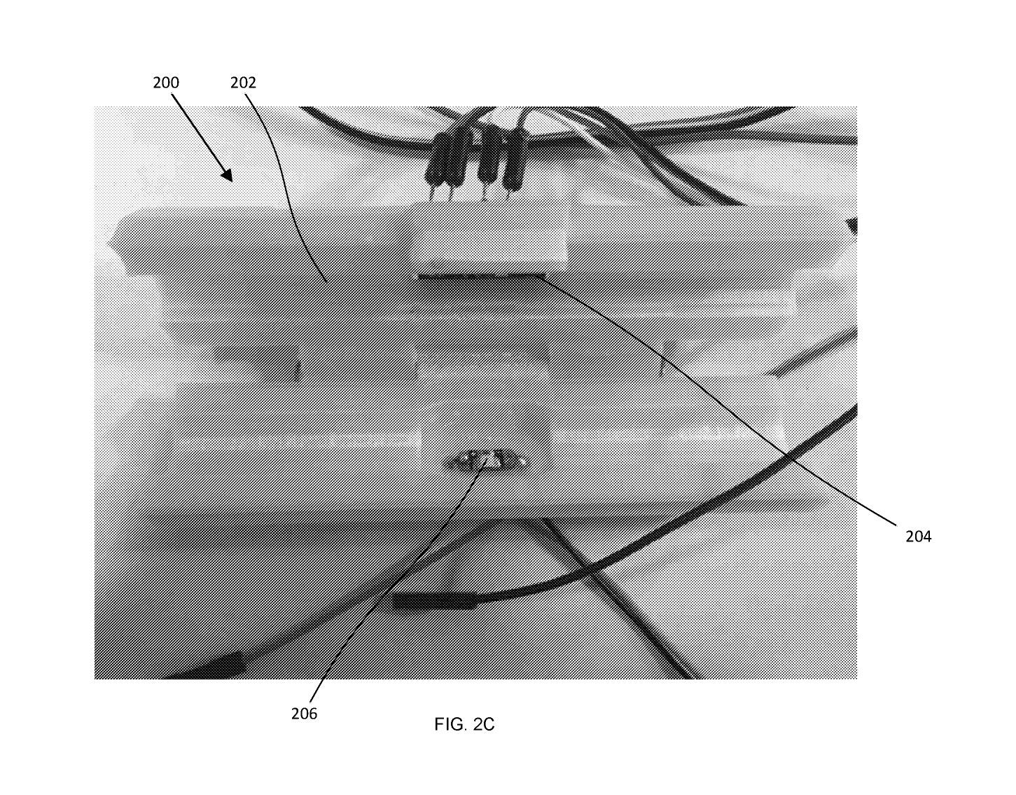

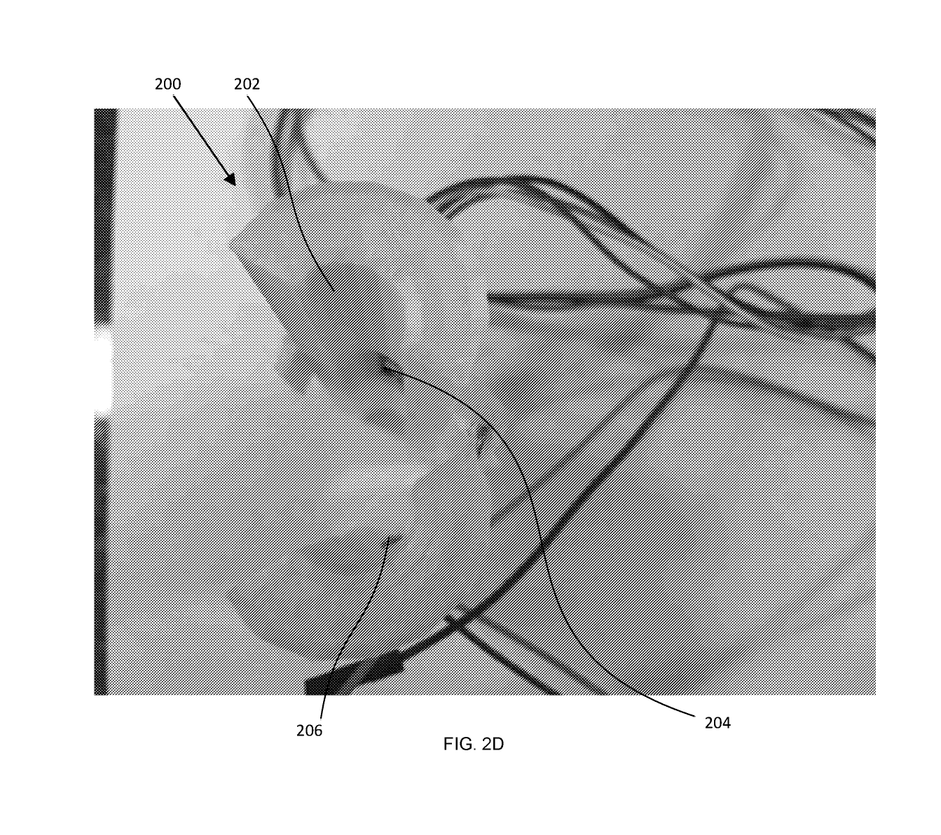

Referring now to FIGS. 2A-2D and FIG. 5, in some embodiments, the device 500 includes a color sensing unit 200, also referred to as a colorimeter. The color sensing unit 200 can be adapted and/or configured for attachment to a clear portion of Foley catheter tubing 112. The color sensing unit 200 includes a color sensor 204, and can actively sense the color and other properties of the fluid that is draining from the patient (i.e., outflow 514 from the catheter 100). The color sensor 204 can be programmed to detect a color of outflow 514 from the catheter 100. The color sensor 204 can then relay information to a controller 504, which can be in communication with other portions of the device 500 (e.g., occlusion unit 400, pressure sensing unit 300, flow rate sensor 502, and the like).

The color sensing unit 200 of the device 500 can be based upon the optical properties of liquid. In one embodiment of the invention, the color sensing unit 200 utilizes a variation of the Beer-Lambert Law, which states that the absorbance of a material sample (in this case, urine/blood or outflow 514) is proportional to the concentrations of the attenuating species in the material sample. Specifically, when applied to the device 500 herein, the higher the concentration of solute in the urine or outflow solution 514 (red blood cells being among these) the more light the urine outflow solution 514 will absorb. By placing a known wavelength and intensity of light across the urine tubing 510, a corresponding photodiode sensor 204 can measure the resulting signal intensity and determine an increased or decreased concentration of solute in the urine or outflow solution 514.

Embodiments of the invention can measure both the color and the intensity of the color, which is indicative of the concentration of light-absorbing particles within the outflow 514. In some embodiments, the color sensing unit 200 includes a light source 206 that applies the full spectrum of visible light and a corresponding photodiode sensor 204. Urine tubing or outflow tubing 510 is passed between the light source 206 and photodiode sensor 204. The photodiode 204 can be configured to detect wavelengths of the light that is not absorbed by a sample (i.e., urine or outflow 514) within the portion of the lumen.

The light source 206 can be any commercially available light source that provides the full spectrum of visible light (e.g., any commercially available bright white light emitting diode (LED)). The photodiode sensor 204 can be any commercially available photodiode.

By detecting color, titration of inflow 516 or outflow 514 can be tuned specifically to blood in the urine (or outflow 514) instead of the overall concentration of the urine solution or outflow 514. For example, a patient may be taking medications that change the urine color and increase the urine concentration and absorbance. Additionally, a patient may have more concentrated urine from dehydration. Such scenarios would confound the aforementioned utilization of the Beer-Lambert law by falsely reading there was more blood in the urine, and unnecessarily waste irrigant and prevent accurate titration. By utilizing the full spectrum of visible light, the device 500 allows for titration specifically for blood in the urine or outflow 514 and more accurate recognition or detection of clots within the tubing 510.

In certain embodiments, the color sensor is a three-channel sensor or an RGB color sensor. In some embodiments, the RGB color reading is in the format (R, G, B), where “R” is the value in the red channel, “G” is the value in the green channel, and “B” is the value in the blue channel. A particular color is determined by the combination of the R,G,B values. For example, in some embodiments, the RGB color sensor 312 reading (0,0,0) corresponds to black, the color reading (65536,0,0) corresponds to pure red, the color reading (0,0, 65536) corresponds to pure blue, and the color reading (65536, 65536, 65536) corresponds to white.

A clot within the tubing 510 can be detected by detecting a significant decrease in transmittance (light cannot pass easily through a clot) and/or a transient drop in all three RGB values from the color sensor 312 to black (0,0,0). Thus, device 500, for example, can be programmed to recognize a clot if transmittance is decreased below a specific threshold or a decrease in transmittance above a specified amount is detected. Device 500 can also be programmed to recognize a clot if the color sensor 312 reading reads black (e.g., (0,0,0)).

During titration of blood, such as in the case of hematuria, blood in the urine can be detected by detecting the color red (e.g., a high value in the red channel relative to the values in the other channels of a RGB color sensor 312). For example, in one embodiment, the maximum pure red reading is (65536,0,0). As the urine turns dark red, the R value drops towards 0 and the whole spectrum approaches black (0,0,0). The device 500 can be programmed to increase the rate of irrigation inflow 516 when the color sensor 312 detects red or black to flush the blood in the urine.

To maintain irrigation efficacy, the values in the green and blue channels can be monitored. Clear urine appears “white” to the color sensor 312 (e.g., a reading of or close to (65536, 65536, 65536) in one embodiment of a color sensor 312). During titration, increases in the values in the green and blue channels indicate the outflow 514 is turning clear or white. Thus, the device 500, for example, can be programmed to decrease irrigation flow 516 when increases in the values in the green and/or blue channels are detected.

Irrigation efficiency can also be maintained by differentiating between possible solutes in the urine. For instance, a patient taking UMBEL® (methenamine, sodium phosphate monobasic, phenyl salicylate, methylene blue, and hyoscyamine sulfate), a medication often used for treating urinary irritation, can have blue-colored urine. In this case, the outflow during a “clear urine scenario” would show pure blue on the color sensor 312 (e.g., a color reading of (0,0,65356) in one embodiment of the color sensor 312). If a patient was taking URIBEL® and also had hematuria, then the color reading from the sensor 312 would show a mix of red and blue (e.g., a reading of (65536,0,65536) in one embodiment of the color sensor 312. Thus, the value in the blue channel would not be useful for detecting clearing of the urine or outflow 514. To detect clearance of the hematuria, the device 500 can be programmed instead to focus on increases in the value of the green channel, rather than the values in the blue channel. In this case, for example, the device 500 can be programmed to decrease irrigation flow 516 when an increase in the value in the green channel is detected.

FIGS. 2A-2D show a prototype color sensing unit (CSU) 200. The design of the prototype utilizes a 3D-printed enclosure of original design that is cylindrical and able to securely fit onto existing Foley tubing 112. A housing 202 can be adapted and/or configured to surround a portion of a lumen such as Foley tubing 112. The housing 202 can be a clamshell mechanism with a hinge (e.g., a living hinge) and/or a clasp for easy placement and removal. The housing 202 can be any other mechanism that enables quick placement and removal of the CSU 200 to and from the tubing 112. The housing 202 can be formed from an optically opaque material in order to shield the internal components from ambient conditions. A color sensor 204 can be positioned on one side of the tubing 112 and an bright white LED light 206 can be positioned on the opposite side. By trans-illuminating the urine or outflow 414 through the tubing 112 in this manner, the photodiode sensor 204 receives a more accurate signal that corresponds to the color of the urine or outflow 514.

### Pressure Sensor Unit (“PSU”)

The device 500 can also include a pressure sensing unit (“PSU”) 300. The pressure sensing unit 300 can be adapted, configured, and/or programmed to continuously monitor bladder pressure and/or provide active feedback to shut off flow to the bladder if, for example, there is a gradual increase in pressure that could signify a clot or other obstruction in the Foley tubing 112.

The PSU 300 provides an important safety mechanism for the device 500. Specifically, this feature allows the device 500 to be truly autonomous. The PSU 300 can connect to any currently available Foley catheter 112 and therefore allows quick and trouble-free integration into any hospital.

The PSU 300 of the device 500 operates on the basis that a Foley catheter 112 placed within a patient's bladder already has an inflated balloon 110. By attaching an appropriate pressure sensor 312 to the balloon port 302 and maintaining a column of incompressible fluid 306 within a tubing 310 connected to the Foley balloon 110, pressure experienced by the balloon 110 within the patient's bladder is sensed by the pressure sensor 312. Embodiments of the invention can measure both absolute changes in the relative pressure within the bladder and the rate of pressure change. Both of these values allow the device 500 to maintain safe pressure levels within the bladder and shutoff titration and/or alert medical personnel if there is a problem.

Pressure sensor 312 can be an electromechanical device that generates an electronic signal based on a pressure applied to or within the sensor 312. For example, the pressure sensor 312 can include a strain gauge (piezoresistive transducer) that allows varying levels of current to flow through the pressure sensor 312 depending on the pressure applied to the sensor 312. Suitable pressure sensors are available from OMEGA Engineering, Inc. of Stamford, Conn. and Freescale Semiconductor, Inc. of Austin, Tex. In some embodiments, pressure sensor 312 has a sensitivity range of 0 to 5 psi.

Pressure sensor 312 can be fluidically coupled to the balloon port 302 of a Foley catheter 112. The pressure sensing unit 300 may be connected to the balloon port 302 using a Luer-lock type fitting 304. A non-compressible column of fluid 306 in the unit 300 would transmit any increases in pressure to the pressure sensor 312. In some embodiments, an increase in pressure triggers cessation of bladder inflow 516. The pressure increase can be transmitted to the controller 504, which can send a signal to the occlusion unit 400 to compress irrigation tubing 508 to reduce or shutoff inflow 516.

In some embodiments, the device 500 is programmed to terminate inflow of irrigant and/or send an alarm to alert medical personnel if the pressure reading from the pressure sensor 312 is above a specified value or if the change in the pressure reading is above a specified amount of pressure variation. Normal changes in physiologic bladder pressure are typically 0-200 cm H₂O (or equivalently, 0-2.84 psi). In some embodiments, the device 500 is configured so as to maintain bladder pressure within a specified range (e.g., a normal physiologic bladder pressure range). The device 500 or pressure sensing unit 300 can be configured or programmed to read or process pressure either as absolute pressure values or changes in pressure relative to a specified pressure. For example, the device 500 can be programmed to reduce or terminate inflow 516 or send an alarm if the reading from the pressure sensor 312 is above a specified value (e.g., 2.5 psi, 3 psi, 3.5 psi, or 4 psi). Alternatively, the device 500 can be programmed to reduce or terminate inflow 516 or send an alarm if the change in pressure is above a certain amount (e.g., 0.1 psi, 0.5 psi, 1 psi, or 2 psi). In some embodiments, the pressure reading from the pressure sensor 312 ranges between 2 to 5 psi.

The pressure sensing unit 300 provides at least several advantages. For example, the pressure sensing unit 300 permits continuous bladder irrigation (CBI) to be used during delicate situations, such as when the bladder is weaker or sensitive to rupture. Ordinarily, if the Foley catheter 100 is unclogged and functioning appropriately, the pressure in the bladder is in steady state and is expected to be close to neutral pressure (i.e., the pressure sensor 312 will simply sense the inherent pressure within the Foley balloon as baseline). If, for example, a clot were to cut off the outflow 514, then the pressure in the bladder would begin to rise as irrigation 516 continues to fill the bladder in a standard CBI setup. As the pressure increases, the patient would begin to experience severe pain and the bladder would be at risk for rupture. This is particularly important in the case of CBI after large trans-urethral tumor resections (TURBT), where the procedure leaves the bladder walls weaker than normal. Because of the risk of bladder rupture, some physicians refuse to use CBI in patients after large TURBTs, and these patients can develop clots and severe discomfort that require significant time for nurses, physicians, and other staff to irrigate and keep clear. In some of these cases, significant bleeding that can normally be managed with CBI can require further surgeries for clot evacuation, which is both time-consuming and expensive. Because the device 500 is able to sense bladder pressure, CBI can be safely used even in such delicate situations. In such situations, an increase in pressure can cause irrigation inflow 516 to shutoff and an alarm to be sent to alert nursing or other medical personnel of potential unsafe pressure.

Another advantage the device 500 provides is the ability to measure or sense rate of change of pressure. Measurement or calculation of the rate of change of pressure provides the ability to discriminate, for example, between a bladder spasm and a true catheter obstruction. Having a catheter 100 in place can cause bladder spasms in a patient. As a patient's bladder senses irritation, the patient suddenly experiences an urge to void and a subsequent involuntary and forceful contraction of the bladder. This results in a significant increase in pressure over the course of milliseconds to seconds. In patients with CBI, sudden spasms can be mistaken for bladder obstruction. Because of the sudden intense pressure in the bladder, irrigation 516 will slow or stop completely and no output will be seen. In some cases, the bladder spasms can take time to relax and can affect the overall bladder muscle tone. In situations involving bladder spasms, the pressure is variable; however, the time scale of the increase in pressure is on the order of seconds at most.

In contrast, a rise in the tonicity of the bladder would be demonstrated over a time scale of minutes to hours. In some situations, an increased bladder tone does not necessitate the need to shut off inflow 516 to the catheter 100 (e.g., when the bladder is filling up with irrigant). Thus, in some embodiments, device 500 is programmed to ensure safe operating conditions by permitting inflow 516 if bladder pressure is below a threshold value and terminating inflow 516 if pressure is above the threshold value.

A clot, on the other hand, would cause a slow steady increase in bladder pressure. The gradual increase in pressure would be similar to a change in tonicity; however, in the case of increased tonicity, the pressure would eventually plateau and achieve a steady state. In the case of a clot or obstruction in the Foley catheter 100, a slow steady increase in pressure over a few minutes or even hours would be observed (depending upon the degree of obstruction). The device 500 can be programmed to monitor both the time scale of the pressure change and absolute value of the pressure changes and adapt the rates of inflow 516 and outflow 514 accordingly, as well as alert personnel if necessary.

Because the device 500 can measure the rate of change of bladder pressure, the device 500 can recognize the difference between a true obstruction (slow increase in pressure over time) versus and sudden spasms (sudden acute rise in bladder pressure). Through the controller 504, the device 500 can then process the pressure information and perform appropriate step(s) of terminating irrigation 516 and alerting nursing in the case of a true obstruction, or continuing to irrigate in the case of a bladder spasm.

Further, the rate of change of bladder pressure can assist in a physician's diagnosis of a patient's condition by providing insight into an underlying cause of patient pain. This can allow the physician to better treat the patient's cause of irritation.

In some embodiments, to clear an obstruction in the catheter or patient's bladder, suction can be provided to the outflow of the catheter 100. A device providing suction (e.g., a vacuum pump) can also be communicatively coupled to the device 500. During suction, pressure can be monitored by the pressure sensing unit 300 and the suction device can be appropriately shut off when the obstruction is cleared (e.g., when the pressure detected by pressure sensor 312 returns to a normal or baseline pressure).

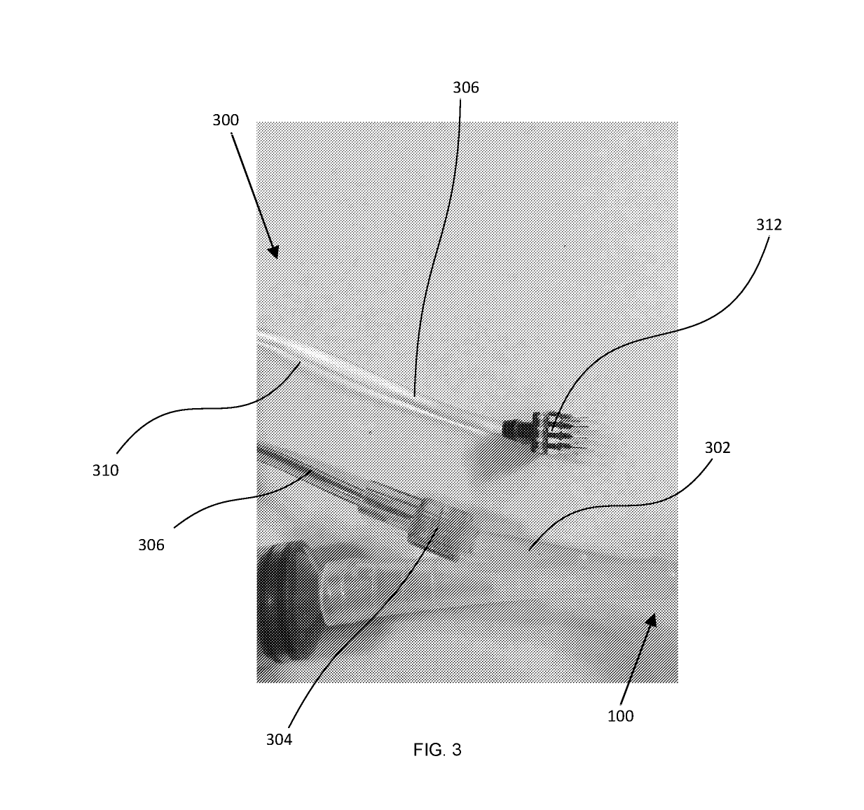

The PSU 300 design in an initial prototype shown in FIG. 3 provides tubing 310 connected on one end to the open mouth of the pressure sensor 312 and on the other to the balloon port 302. The tubing 310 is connected to the balloon port 302 using a Luer-lock type fitting 304. This connection would be watertight and contiguous, with minimal air within the system.

### Occlusion Unit (“OU”)

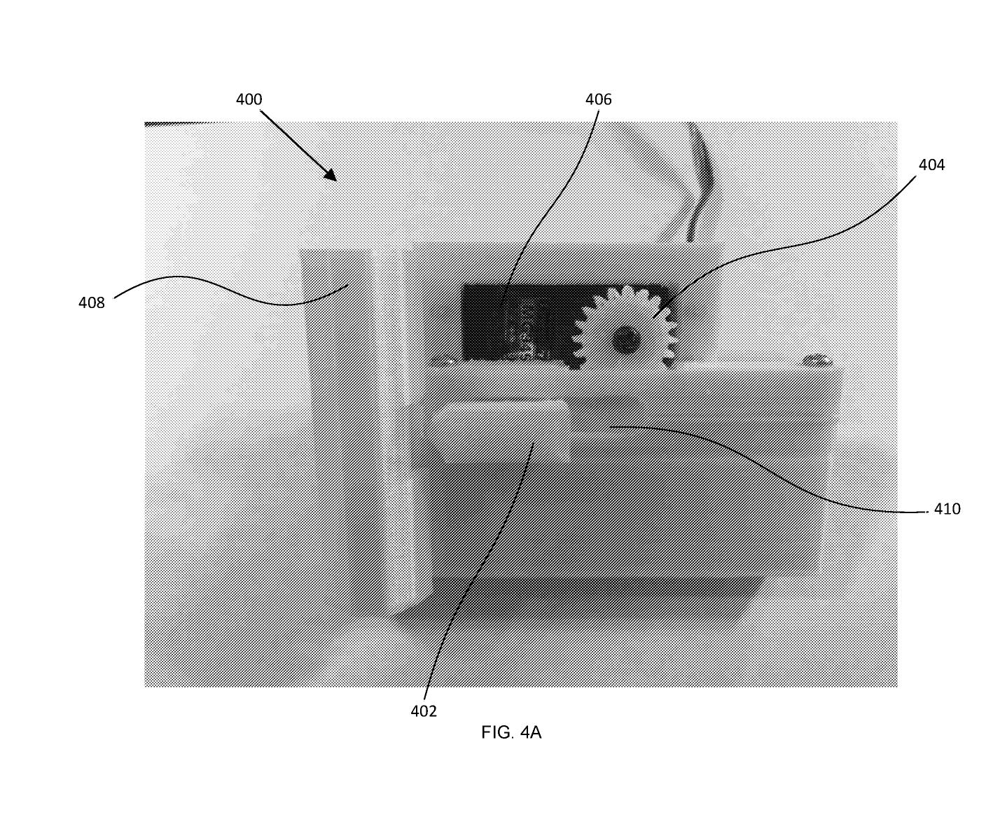

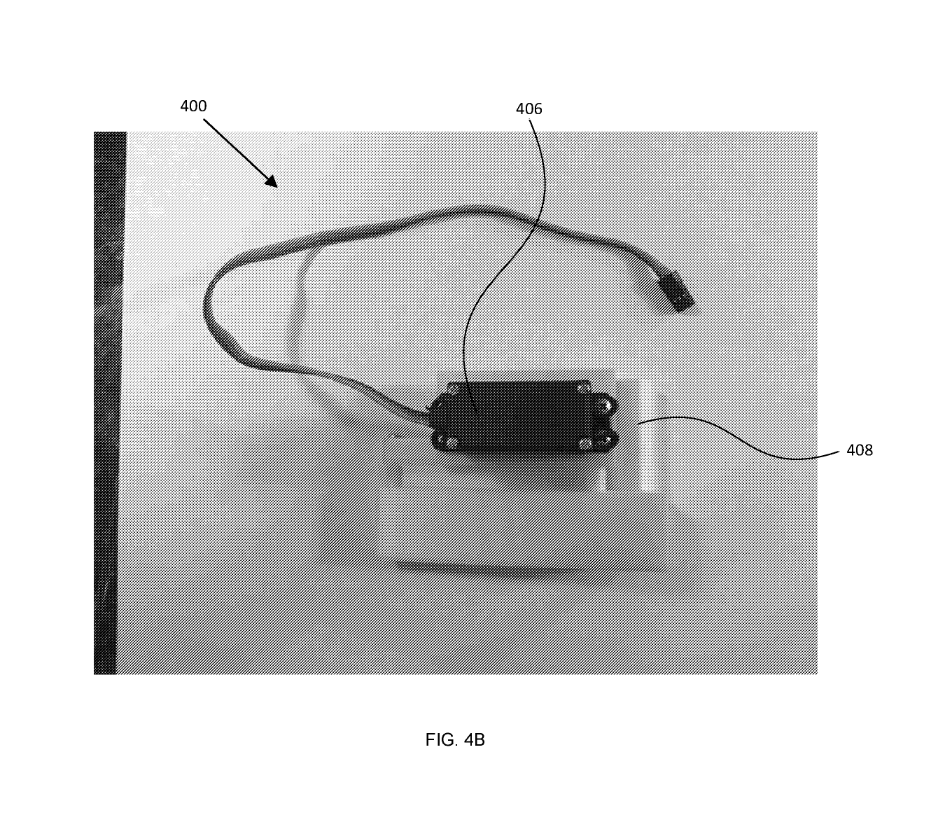

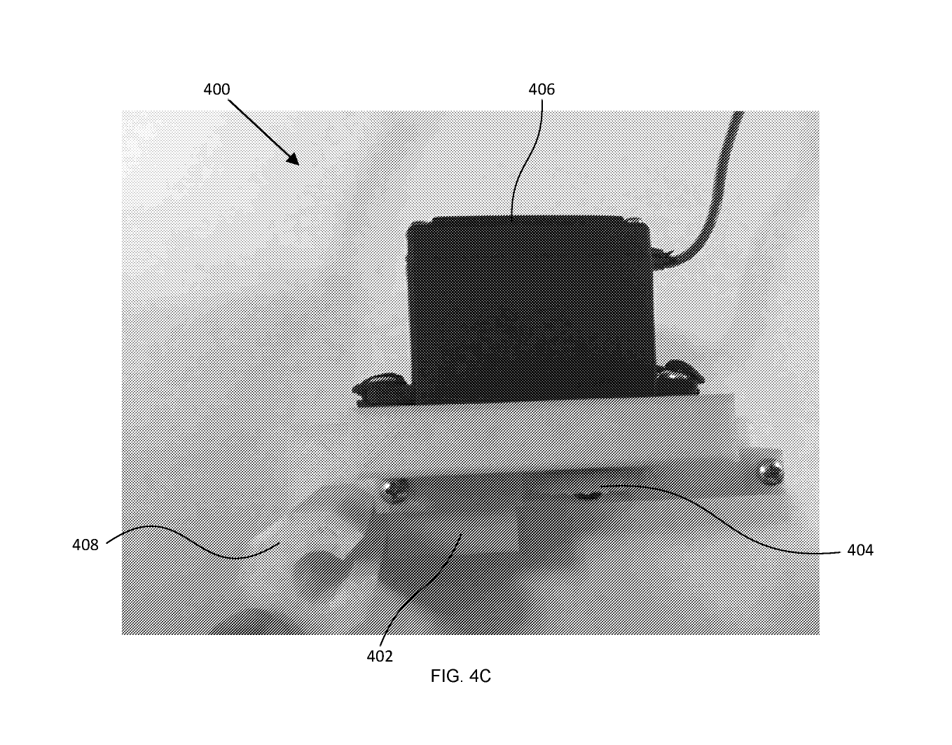

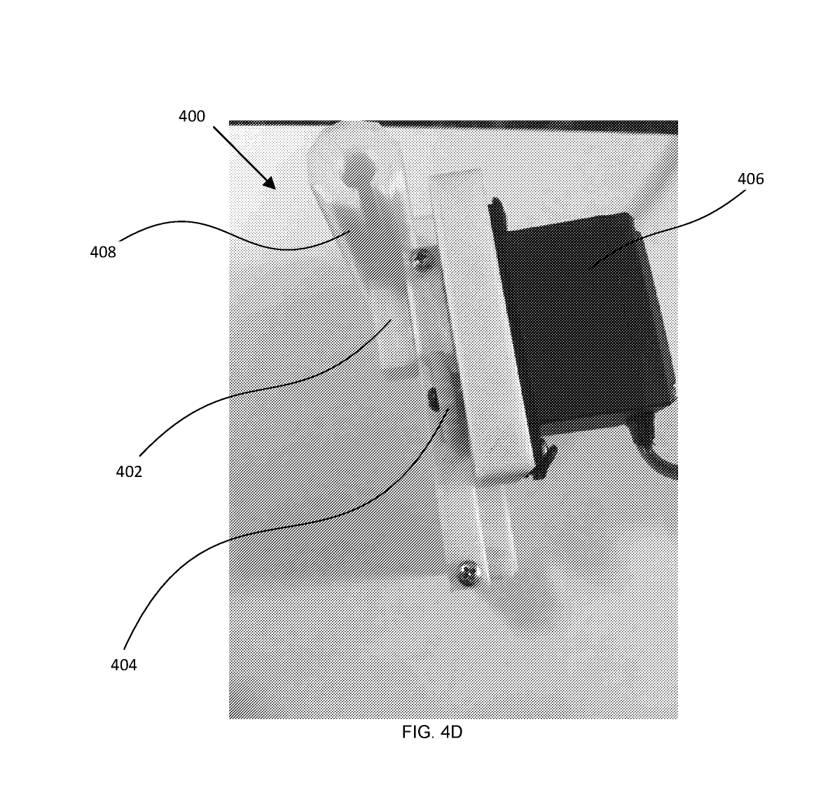

Referring now to FIGS. 4A-4D and FIG. 5, another aspect of the invention provides an occlusion unit (“OU”) 400. The OU 400 is programmed to adjustably occlude a lumen (such as catheter tubing 112, 508) providing irrigation to the catheter. The OU 400 adjusts the inflow of irrigation 516 into the catheter by compressing or releasing irrigation tubing 508. The OU 400 can serve a safety function by suspending inflow when bladder pressure increases to unsafe levels. The OU 400 can also function to adjust the flow rate of inflow for active titration during normal use.

Based on the color of the urine or outflow 514 and/or based on the pressure sensed by the pressure sensor 312, the controller 504 sends a signal to the occlusion unit 400 that actuates an occlusion member to controllably impede flow through the tubing. For example, the occlusion unit 400 can activate a rotational servo motor 406 that progressively compresses or releases the irrigation tubing 508 for the desired effect. The servo motor 406 may be any commercially available servo motor, such as servo motors available under the SMAKN® trademark. In one embodiment, the servo motor 406 is SMAKN® High Torque Metal Gear MG945.

In some embodiments, the servo motor 406 is attached to a structure providing sufficient structural integrity to house the motor. FIGS. 4A-4D show a prototype OU 400 featuring a 3D-printed enclosure of original design that provides a tube 408 that snuggly fits around the standard irrigation tubing 508. In the prototype, a large area of 3D printing material below a linear gear rail provides the structural integrity to house a high torque servo motor 406.

In some embodiments, the OU 400 includes one or more gears 404 attached to the servo motor 406 and configured to move a movable member (such as a piston 402 or a cam) to impinge a lumen and occlude flow of liquid in the lumen. In the prototype shown in FIGS. 4A-4D, for example, the high torque servo motor 406 is attached to a circular gear 404 and a corresponding linear gear within the gear rail that converts rotational motion to linear motion. Upon activation of the servo motor, a piston 402 connected to the linear gear is forced to mechanically compress the tubing 508. The circular gear 404 and linear gear move piston 402 along a fixed track 410 to occlude the inflow tubing 508 that passes through column 408. Column 408 is adapted and/or configured to surround a portion of a lumen such as Foley tubing 112. The servo motor 406 can rotate through a 180 degree arc, and can be controlled to a single degree. By manipulating the servo motor 406 one (1) degree at a time, the flow rate of irrigation 416 that is provided to the bladder through the tubing 408 can be controlled.

In another embodiment, an alternate mechanism involving a continuous rotation servo motor is provided. The continuous rotation servo motor moves circular gears that are attached to a threaded length of metal that can effectively and more accurately compress and release the tubing. This embodiment is expected to be more durable, as the gears in the initial prototype experience very high resting torque during normal usage and can fatigue.

### Flow Rate Sensor (“FRS”)

In some embodiments, the device 500 includes a flow rate sensor 502. The device 500 can also include a flow regulation valve. In some embodiments, the device 500 includes an outflow valve. The flow rate sensor 502 can be adapted, configured, and/or programmed to measure the flow of irrigant 516 to the catheter 100 (and, by extension, into a patient). The flow rate data can also be used as feedback to actively titrate irrigant flow 516 and predict when irrigant in the current irrigation bag 506 will be depleted so that nursing staff or other healthcare providers can be appropriately alerted.

As shown in FIG. 5, the device 500 can contain a flow rate sensor 502 and a means for controlling or adjusting flow rate, such as the occlusion unit 400. In some embodiments, the device 500 can further include a pump coupled to the irrigant flow so that irrigant inflow rate can be controlled or adjusted by controlling the pump. The pump can also be communicatively coupled to the controller and/or other units of the device 500, such that, for example, the pump can be activated or shut off in response to a specified pressure or color of outflow 514 detected.

Any commercially available flow rate sensor 502 can be added between the irrigation bag 506 and tubing 508 to allow the device 500 to measure how much fluid from any given bag 406 has already run through. In some embodiments, the flow rate sensor 502 is positioned below the occlusion unit 400 on the irrigation tubing 508. The flow rate sensor selected is preferably configured and adapted to measure flow rate in the irrigation tubing while minimizing any disruption of the irrigation flow to the catheter and any contamination of the irrigation. Suitable flow rate sensors 502 include, for example, clamp-on ultrasonic flow meters for small tubes.

The portion of the device 500 comprising the flow rate sensor 502 and occlusion unit 400 (or other similar means for controlling flow rate of inflow 516, such as a pump) can be adapted, configured, and/or programmed to measure the flow of irrigant 516 into a patient, actively titrate that flow 516, and/or passively track and predict when irrigant in the current irrigation bag 506 will be depleted so that nursing staff or other healthcare providers can be alerted accordingly. In some embodiments, the device 500 is configured to alarm when the irrigation bag 506 needs to be changed so that the likelihood of the irrigation running out completely is minimized. This minimizes the risk of the patient's bladder clotting off due to disruption of the irrigation 516 prematurely.

### Controller

Referring now to FIG. 5, in some embodiments, the device 500 includes a controller 504 in communication with the various units or portions 200, 300, 400, 502 of the device 500, such as the color sensing unit 200, pressure sensing unit 300, or occlusion unit 400. Any one or more of the units can be communicatively coupled to the controller 504 and/or to another unit in a feedback loop to maintain, for example, a specified color of the urine or outflow 516 or specified flow rate. In one embodiment, the controller is in communication with the color sensor 204 and occlusion unit 400 and is programmed to control the occlusion unit 400 to titrate irrigation flow 516 to the catheter based on the color detected by the color sensor 204 in order to achieve a specified color, spectrum, or range of outflow 514 from the catheter.

Controller 504 can be an electronic device programmed to control the operation of other components to achieve a desired result. The controller 504 can be programmed to autonomously carry out a CBI regimen without the need for input (e.g., from medical professionals) or can incorporate such inputs. The principles of how to use feedback (e.g., from a color sensing unit 200 and/or a pressure sensing unit 300) in order to modulate operation of a component such as an occlusion unit 400 are described, for example, in Karl Johan Astrom & Richard M. Murray, Feedback Systems: An Introduction for Scientists & Engineers (2008).

In one embodiment, device 500 can include an electronic controller or microcontroller 504 programmed to monitor, report, and/or control the operation of device 500. Such a controller 504 can be fabricated using a variety of electronics architectures such as an ARDUINO® or IOIO™ microcontroller. The controller 504 can be coupled to one or more power sources (e.g., one or more batteries such as lithium polymer batteries), memory and/or storage devices (e.g., a micro SD card), pressure sensors 212, flow meters or flow rate sensors 402, occlusion unit 300, color sensors 204, display devices 314 (e.g., a liquid crystal display), and pumps. Memory and/or storage devices can be suitable for storing data as well as instructions for programmed processes for execution on a processor. Controller 504 can contain or load one or more computer-readable program instructions for implementing one or more algorithms to maintain a desired outflow 516 color, flow rate, and the like. In some embodiments, a user (e.g., a healthcare provider) can adjust one or more parameters of the system. In other embodiments, the controller 504 can communicate, through wired or wireless protocols, with a general-purpose computer, a tablet computer, a smartphone, and the like.

In some embodiments, the controller 504 can include the appropriate hardware and/or software to implement one or more of the following communication protocols: Universal Serial Bus (USB), USB 2.0, IEEE 1394, Peripheral Component Interconnect (PCI), Ethernet, Gigabit Ethernet, and the like. The USB and USB 2.0 standards are described in publications such as Andrew S. Tanenbaum, Structured Computer Organization Section § 3.6.4 (5th ed. 2006); and Andrew S. Tanenbaum, Modern Operating Systems 32 (2d ed. 2001). The IEEE 1394 standard is described in Andrew S. Tanenbaum, Modern Operating Systems 32 (2d ed. 2001). The PCI standard is described in Andrew S. Tanenbaum, Modern Operating Systems 31 (2d ed. 2001); Andrew S. Tanenbaum, Structured Computer Organization 91, 183-89 (4th ed. 1999). The Ethernet and Gigabit Ethernet standards are discussed in Andrew S. Tanenbaum, Computer Networks 17, 65-68, 271-92 (4th ed. 2003).

In other embodiments, controller 504 can include appropriate hardware and/or software to implement one or more of the following communication protocols: BLUETOOTH®, IEEE 802.11, IEEE 802.15.4, and the like. The BLUETOOTH® standard is discussed in Andrew S. Tanenbaum, Computer Networks 21, 310-17 (4th ed. 2003). The IEEE 802.11 standard is discussed in Andrew S. Tanenbaum, Computer Networks 292-302 (4th ed. 2003). The IEEE 802.15.4 standard is described in Yu-Kai Huang & Ai-Chan Pang, “A Comprehensive Study of Low-Power Operation in IEEE 802.15.4” in MSWiM'07 405-08 (2007).

Controller 504 can communicate with various telemetry systems used in hospitals for centralized monitoring of patients within a unit. For example, controller 504 can send secure messages to healthcare professionals regarding status, measurements, reports, alerts, alarms, and the like.

In one embodiment, the controller 504 is a commercially available ARDUINO® all-purpose/open source microprocessor. Such a microprocessor has agnostic integration with various sensors, open source hardware and software design, and adequate power for testing and expanding. Any embodiments of the device 500 can use custom boards in order to tailor power and sensor requirements exactly.

In one embodiment, the controller 504 is programmed with custom C++ code. The program receives pressure readings from the PSU 200 as input. If the pressure is within range of normal, the program activates the CSU 200 and begins to read the color. Based upon the range of color detected, a preprogrammed algorithm progressively opens and closes the occlusion unit 300. The program is designed to titrate the drip based upon threshold values of colors, with the overall goal being to use the least amount of irrigation possible. The CSU 200 is programmed to run at multiple data points per second. This can be varied to conserve power and make the system less sensitive to small variations. In some embodiments, the controller 504 provides appropriate alarms needed to alert of any issues (e.g., detection of catheter obstruction or unsafe pressure).

Controller 504 can, thus, provide for executing processes, by itself and/or in cooperation with one or more additional devices, that can include algorithms for controlling occlusion unit in accordance with the present invention. Controller 504 can be programmed or instructed to perform these processes according to any communication protocol and/or programming language on any platform. Thus, the processes can be embodied in data as well as instructions stored in a memory device and/or a storage device or received via a user interface and/or communication interface for execution on a processor.

Controller 504 can control the operation of the occlusion unit in a variety of ways. For example, controller 504 can modulate the level of electricity provided to the occlusion unit 400. Alternatively, the controller 504 can transmit instructions and/or parameters to the occlusion unit 400 for implementation by the occlusion unit.

### Method of Monitoring Bladder Pressure

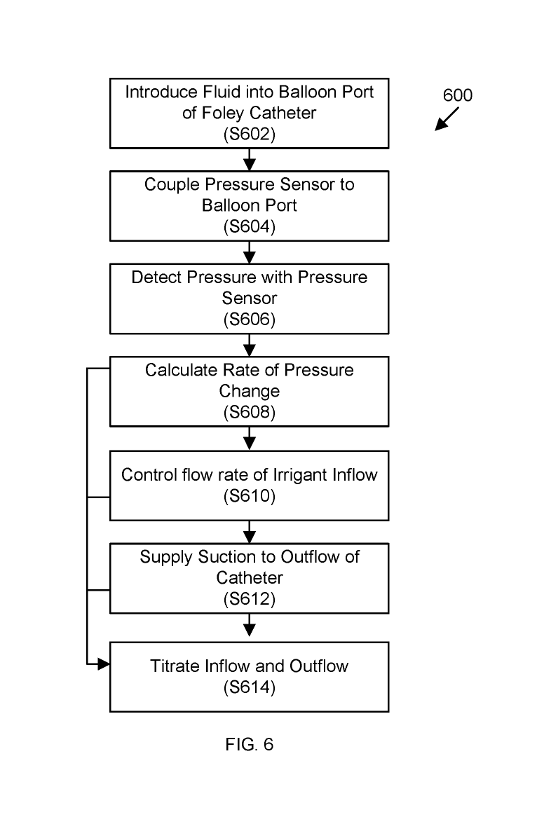

Referring now to FIG. 6, another aspect of the invention provides a method 600 of monitoring bladder pressure. In one embodiment, the method 600 includes the step S602 of introducing a fluid into a balloon port 106, 302 of a Foley catheter 100 to inflate a balloon 110 of the Foley catheter 100 while the balloon 110 is naturally resting within the bladder. Inflation of the balloon 110 keeps the Foley catheter 100 in place within the patient.

The method 600 includes the step S604 of coupling a pressure sensor 312 to the balloon port 302. For example, the pressure sensor 312 can be fluidically coupled to the balloon port 302 by connecting a tubing 310 to the pressure sensor 312 on one end and to the balloon port 302 on the other end. The tubing 310 can be connected to the balloon port 302 using a Luer-lock type fitting 304. This connection should be watertight and contiguous, with minimal air within the system.

In step S606, pressure exerted on the Foley balloon through a column of fluid in contact with the pressure sensor 312 is detected. The pressure detected is the pressure experienced by the balloon 110 within the patient's bladder. The column of fluid can be any incompressible fluid, such as water.

In some embodiments, the method 600 further includes the step of calculating S608 a rate of pressure change. Measuring the rate of pressure change allows for example, detection of the difference between a bladder spasms and a true catheter obstruction. Recognizing the difference between a true obstruction (slow increase in pressure over time) versus sudden spasms (sudden acute rise in bladder pressure) allows appropriate step(s) to respond to the situation to be performed, e.g., shutting off irrigation 516 and alerting nursing or medical personnel, or simply ignoring the bladder spasm and continuing to irrigate.

In still other embodiments, the method 600 further includes any one or more of the steps of controlling the flow rate of irrigant inflow 516 (S610), supplying suction to outflow of the catheter (S612), or titrating inflow and outflow through a controller 504 (S614). In situations of increased or unsafe bladder pressure, for example, the flow rate of irrigant inflow can be reduced or shut off. In some embodiments, the inflow is controlled or actuated by a pump mechanism.

Suction to the outflow of the catheter 100 can be provided in order to evacuate or clear obstructing matter within the catheter or bladder. The outflow and inflow can be titrated through the controller 500 to permit fine control of bladder pressure and generation of a desired pressure.

The calculating, controlling, and/or titrating steps S608, S610, S614 can be implemented in computer-readable program instructions that can be stored on computer-readable media for loading and/or execution by a processor.

The invention described herein addresses the need for more efficient and safer continuous bladder irrigation (“CBI”) systems and methods. The invention would decrease the overall time of CBI therapy needed per patient, decrease the amount of irrigation used, decrease burden on nursing staff and increase autonomy, decrease complication rate when using CBI. The Autonomous Continuous Bladder irrigation Device (ACBID) 500 provided by the invention is designed to completely regulate and automatically provide safe and efficient continuous bladder irrigation therapy to patients with hematuria. The device 500 is further designed to be completely compatible with all existing irrigation tubing, Foley catheters, and Foley tubing. No other device that is able to automate CBI is believed to exist. Additionally, the bladder pressure sensing method described herein is believed to be novel. The device 500 described herein is expected to revolutionize therapy for patients with hematuria, transforming CBI into a safe, efficient, and low burden therapy option.

### Measurement of Blood Loss

Embodiments of the invention can measure blood loss. As discussed herein, the amount of blood in a urine stream can be measured using color sensor 312 based on the intensity and color of the received signal using the Beer-Lambert Law. The measured concentration of blood in a urine stream can be multiplied by the measured volume of urine passed to determine the amount of blood lost via urine. For example, the product of instantaneous flow rates as measured by flow rate sensor 502 and instantaneous concentration measured by color sensor 312 can be integrated over time to measure cumulative blood loss via urine.

### Exemplary Applications

Embodiments of the invention can be applied or a variety of purposes such as bladder pressure monitoring and bladder irrigation. Examples of situations where bladder pressure monitoring may be desired include trauma, therapeutic hydrodistention, and neurogenic bladder monitoring in spinal cord patients. Examples of situations where bladder irrigation for hematuria may be desired include malignancy or radiation inflammation.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. An autonomous continuous bladder irrigation device comprising:
a color sensor programmed to detect a color of outflow from a catheter;
an occlusion unit programmed to adjustably occlude a lumen providing irrigation to the catheter; and
a controller in communication with the color sensor and the occlusion unit and programmed to control the occlusion unit to titrate irrigation flow to the catheter based on the color detected by the color sensor in order achieve a specified color of outflow from the catheter.

2. The device of claim 1, further comprising:
a pressure sensor adapted and configured to couple with a balloon port of the catheter.

3. The device of claim 2, wherein the controller is in communication with the pressure sensor and is further programmed to control the occlusion unit to cease irrigation flow in response to a pressure measurement.

4. The device of claim 1, wherein the occlusion unit includes a movable impingement member adapted and configured to impinge flow through the lumen.

5. The device of claim 4, wherein the impingement member can be a piston.

6. The device of claim 4, wherein the impingement member is selected from the group consisting of a cam.

7. The device of claim 4, wherein the occlusion unit includes a servo motor or a stepper motor adapted and configured to move the impingement member.

8. The device of claim 1, further comprising:
a flow rate sensor in communication with the controller, the flow sensor adapted and configured to measure a flow rate within the lumen below the occlusion unit.

9. The device of claim 1, wherein the color sensor comprises:
a housing adapted and configured to surround a portion of a lumen;
a light source positioned within a first side of the housing and adapted and configured to apply light to the portion of the lumen; and
a photodiode sensor positioned on a second, opposite side of the housing and adapted and configured to detect wavelengths of the light that is not absorbed by a sample within the portion of the lumen.

10. The device of claim 1, wherein the housing comprises a first half and a second half rotatably coupled by a hinge.

11. A colorimeter comprising:
a housing adapted and configured to surround a portion of a lumen;
a light source positioned within a first side of the housing and adapted and configured to apply light to the portion of the lumen; and
a photodiode sensor positioned on a second, opposite side of the housing and adapted and configured to detect wavelengths of the light that is not absorbed by a sample within the portion of the lumen.

12.-14. (canceled)

15. A method of monitoring bladder pressure, the method comprising;
introducing a fluid into a balloon port of a Foley catheter to inflate a balloon of the Foley catheter while the balloon is naturally resting within the bladder;
coupling a pressure sensor to the balloon port; and
detecting the pressure exerted on the Foley balloon through a column of fluid in contact with the pressure sensor.

16. The method of claim 15, wherein the fluid is a substantially incompressible fluid.

17. (canceled)

18. The method of claim 15, further comprising:
calculating a rate of pressure change.

19. The method of claim 15, further comprising:
controlling an irrigant inflow rate.

20. The method of claim 19, wherein the irrigant inflow rate is controlled by actuation of a pump mechanism.

21. The method of claim 20, further comprising
applying suction to outflow of the catheter.

22. The method of claim 21, wherein the suction applied to the outflow of the catheter evacuates obstructing matter within the catheter or bladder.

23. The method of claim 15, further comprising:
titrating outflow from and inflow to the catheter.

24. The method of claim 23, wherein the outflow and inflow are titrated through a controller to generate a specified pressure using the detected pressure as feedback.
